(19) United States
(12) Patent Application Publication
(10) Pub. No.: US 2016/0198987 A1
(43) Pub. Date: Jul. 14, 2016

(54) A DEVICE AND A SYSTEM FOR MEASURING AND PRESENTING BLOOD GLUCOSE CONCENTRATION

(71) Applicant: MODZ OY, Helsinki (FI)

(72) Inventors: Salla KOSKI, Espoo (FI); Carina RAJALA, Espoo (FI); Mikko TASANEN, Espoo (FI)

(73) Assignee: MODZ Oy, Espoo (FI)

(21) Appl. No.: 14/913,242

(22) PCT Filed: Aug. 21, 2014
(86) PCT No.: PCT/FI2014/050643

§ 371 Date: Feb. 19, 2016

(30) Foreign Application Priority Data
Aug. 21, 2013 (FI) 20135852

(51) Int. Cl. A61B 5/145; A61B 5/00; G06F 19/00

(52) U.S. Cl. CPC A61B 5/14532; G06F 19/3406; G06F 19/322; A61B 5/7475; A61B 5/742; A61B 5/7405

(57) ABSTRACT

A measurement device and a system for measuring and presenting data which relates to human health are especially applicable for e.g. monitoring and care of diabetes disease. The purpose is to present clearly and in motivating manner a blood glucose concentration on a displ ay device. In a preferred embodiment, the measurement and monitoring device further includes elements for performing an action in order to present the measured blood glucose concentration result to the user, wherein the action includes coupling of a character expression with a color, a sound and a numerical value.

### TECHNICAL FIELD

An object of the invention is a measurement device and a system for measuring and presenting data which relates to human health. The invention is especially applicable for e.g. monitoring and care of diabetes disease.

### BACKGROUND TECHNOLOGY

A person with a diabetes disease must take especially good care for his/her diet, general condition, regular rest and medication. The care of the diabetes disease also demands regular concentration measurement of blood sugar i.e. blood glucose. The measurement must b performed even several times a day. The measurement commonly takes place by taking a small sample of blood from a fingertip, which sample is let to absorb into a sample test dot of a sample test strip. The glucose concentration is measured from the strip with a portable measurement device, for example. The sample test strip is placed to the measurement device, and a blood sample is put into the sample test strip, after which the measurement device performs blood glucose measurement from a blood sample situated at the sample test dot, and shows the measurement result on the digital display of the measurement device. When a person knows the glucose concentration he/she may assess, whether he/she has need for an instant care action. If the measured blood glucose concentration is too high, e.g. more than 15 mmol/l, the care action is generally adding insulin to blood by injection. If the measured blood glucose concentration is too low, e.g. less than 3 mmol/l, the care action is generally taking drink or food which contains carbon hydrate. In addition to assessing an instant care action the blood glucose concentration gives information on requirement of eating, physical exercise, rest or medication.

In addition to measurement devices of blood sugar concentration there are also programs to be installed in a personal computer, with which a person can monitor the development of blood glucose concentration. When using the program, the measured data can be input to a computer for an analysis performed by the program.

In spite of the fact that portable measurement devices and care equipment are available for caring diabetes, the monitoring and care is insufficient for a large portion of people suffering diabetes. This is caused by the following exemplary factors:
Children and young people do not understand or remember the matters relating to diabetes care and they are not interested in them. On the other hand, parents do not have a possibility to provide persistent looking after the care of the diabetes of a child. Therefore, following a care program would also require a child's high motivation and interest in the care.
Many users are not able to use the available measurement devices and they can possibly not assess the actions that are required based on the measurement results, or to follow the program and time table related to the care.
The doctor taking care of a person and parents of a child do not always have enough information on realization of the care and of the development and the variation of the glucose values.

There also exist systems, by which it is possible to transfer measurement results of the measurement devices to user's work station and possibly further to personnel of the health care, and to perform analyses on the information. Such systems give information on the history of the measurement results, but they do not enhance the motivation of a user for daily care.

In addition, motivating of children to take care of his/her blood sugar can be difficult. Children can have problems to understand numerical values and they might experience the measuring procedure difficult and laborious.

Since diabetes is quite a common disease, insufficient care is a significant problem except for the person himself/herself but also for the society. Therefore, the improvement of care and solving the above problems is important.

### SUMMARY OF THE INVENTION

It is an object of the present invention to implement such a solution, that previously mentioned drawbacks of the prior art could be diminished. In particular, the invention is implied to solve how to present clearly and in motivating manner blood glucose concentration on a display device.

The objective of the invention is met by the features disclosed in the independent patent claims.

A measurement and monitoring device for measuring and presenting blood glucose concentration according to the present invention is characterized by the features of claim 1.

According to an embodiment of the invention, the measurement and monitoring device for measuring and presenting blood glucose concentration comprises measurement means for measuring blood glucose concentration, and a user interface for presenting to a user information which is based on a measurement result, the user interface comprising at least one display and/or means for forming sound. The measurement and monitoring device further comprises means for performing an action in order to present the measured blood glucose concentration result to the user, wherein the action comprises coupling of a character expression with a color, a sound and a numerical value.

In another embodiment, blood glucose concentration results are classified into five different blood glucose concentration categories. This feature is advantageous, because in this way the user has a chance to act before the blood glucose concentration level gets too high or too low. In this embodiment, the user, such as a child, does not have to understand the numerical values of blood glucose concentration, but the category may reliably indicate to him/her if immediate action is required.

In an embodiment, presented character expression, color and sound are related to blood glucose concentration categories. In this way, the blood glucose concentration category based on the measurement result can be presented to the user by using several different ways, which may help the user to identify the blood glucose concentration category in question more reliably and quickly.

In an embodiment, the character expression is selected from numerous character expression categories. This feature may help the motivation of especially children to use the device and it may help children to aim for good blood glucose concentration category and to more easily remember measurement results.

Some preferable embodiments of the invention are described in the dependent claims.

### SHORT DESCRIPTION OF THE DRAWINGS

Next, the invention is described in more detail with reference to the appended drawings, in which

FIG. 1 illustrates a block diagram of an exemplary measurement device according to the invention for the measurement and monitoring of blood glucose concentration;

FIG. 2 illustrates a perspective view of an exemplary measurement device according to the invention;

FIG. 3 illustrates a block diagram of an exemplary system according to the invention for measurement and monitoring of blood glucose concentration;

FIGS. 4a-4e illustrate exemplary character expressions coupled with a specific colors (not shown) relating to different blood glucose concentration categories;

FIGS. 5a-5b illustrate exemplary views of a user interface of the system according to the present invention.

### DETAILED DESCRIPTION OF THE EMBODIMENTS

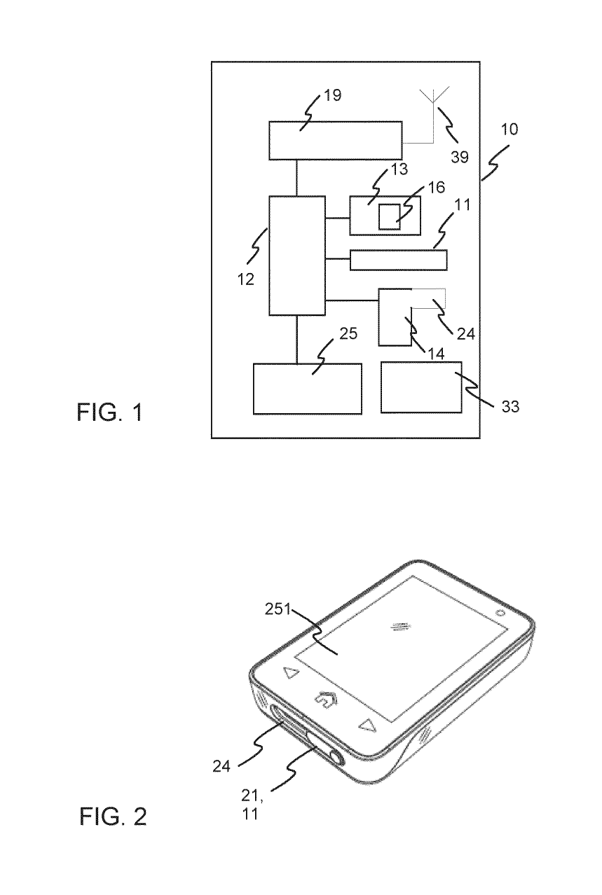

FIG. 1 illustrates a block diagram of an exemplary measurement device according to the invention. The measurement device 10 includes a measurement unit 14 for measuring a blood sample. In the measurement device illustrated in FIG. 1, the measurement unit 14 measures blood glucose concentration from a blood sample which is absorbed into a sample test dot. The measurement unit has an input aperture 24 for placing a test strip, on which a blood sample is then absorbed. The measurement unit is preferably based on electrical charge measurement in a manner, which is prior known as such. The measurement unit preferably includes an amplifier, an analogue-digital converter and other required electronics so that the signal received from the sensor can be fed to the input of a processor 12 of the device. The processor preferably saves the measurement results into a memory 13 for later use. The measurement device may have a single processor, or it may have two or several processor, such as a main processor and an auxiliary processor. In this case the measurement can be performed using an auxiliary processor, which transfers the data to the main processor. The main processor then handles storing and displaying the data.

Also programs 16 controlling the processor have been stored into the memory 13 of the measurement device. Further, data relating to the care program of the user is stored, which data is preferably user specific. Especially, data relating to performing a blood concentration result to a user can be stored in the memory 13. Such data may include blood glucose concentration categories together with one or character expressions, color data and sound data for each blood glucose concentration category. The memory can also comprise other data, such as performance level steps, number of current user performance points and current level.

The measurement device also has user interface means 25, which preferably comprise a display, such as a touch screen. Measurement results can be displayed on the touch screen in text/numbers and/or illustrated with avatars or symbols, for example. The user interface of the measurement device preferably also has means for producing voice and/or vibration. They may produce tones or speech, by means of which the user is guided and given information. The audio signals corresponding to tones or speech can be preferably formed in the processor by means of data stored in the memory. It is also possible that a user can store the figures/sounds used by the device in different performance levels. The user interface may also include other input means, such as press button switches, in addition to the touch screen input.

The measurement device has data transfer port 11, by means of which it is possible to transfer data between the measurement unit and a computer or other equipment of a user. Through the data connection it is possible to transfer measurement results and other user data which are stored by the measurement device to terminal equipment and/or to transfer programs or user data from the computer to the measurement device. The data transfer takes place in a wired manner, e.g. via a micro-USB (Universal Serial Bus) port/connector. Such an electrical connector may be used for other purposes as well, such as connecting to other measurement devices, or charging the battery of the device with a charger or from a USB connection of a computer, for example. It may also be possible that the connection 11 of the measurement device can connect to a data transfer network, whereby it is possible to transfer data with another device which is in connection via the network.

Additionally, the measurement device 10 includes preferably one or several wireless data transfer units 19. If radio data transmission is used the data transfer unit includes an antenna 39. Then the data transfer unit may be e.g. GSM 3G or 4G module of a cellular data transfer system to which a SIM card of a user may be connected. Such a unit may include a specific processor for controlling the data transfer. The communication capability in a mobile cellular system can be used, for example, for transferring measurement data and other data from a measurement device to a mobile phone. Also the data relating to presenting measurement results to the user can be communicated between a server and the user device using this communication capability. If a user is a child, the parents may receive the measurement data to their mobile phones. Also, if a user is an elderly person the relatives of the person may receive the measurement data.

The measurement device may also have a communication unit for Bluetooth communication, for example. Such wireless communication can be used with other measurement devices, such as a device measuring movement of the user or a device measuring heart pulse in order to receive other measurement data. This other measurement data can then be used as a further input in user's care program and it can also be used as the basis of performance levels. However, it may be possible to connect such devices to the wired electrical connection 11 as well.

The data transfer means 11 and 19 may also transfer data between the measurement device and care devices. For example, a measurement device can transmit measurement data to an insulin injector and/or receive from an insulin injector information that the user has received an insulin dose. The measurement device may also use this information as an acknowledgement for the given activity instruction and as information which affects the reminders and activity instructions according to the care program of the user.

As described above, the measurement device may give reminders for the user. The time table for providing the reminders as well as figures and sounds for providing reminders are stored in the memory 13 of the measurement device. The reminders may relate to performing a blood sample measurement, taking a dose of insulin, having a meal, having physical exercise, and resting, for example. The figures and/or sounds of such reminders are preferably related to the performance level of the user. A user may also have a possibility to store such figures/sounds to be used on selected levels.

Based on the measurement results, the measurement device may give an instruction for eating carbon hydrates or taking a dose of insulin. The processor 12 performs the appropriate reminders according to the stored time table. The user acknowledges the reminders and instructions with the corresponding input at the touch screen of the device, for example.

The device of FIG. 1 also includes an energy source 33, such as a recharge-able or disposable battery. A rechargeable battery may be charged via the USB connection, for example.

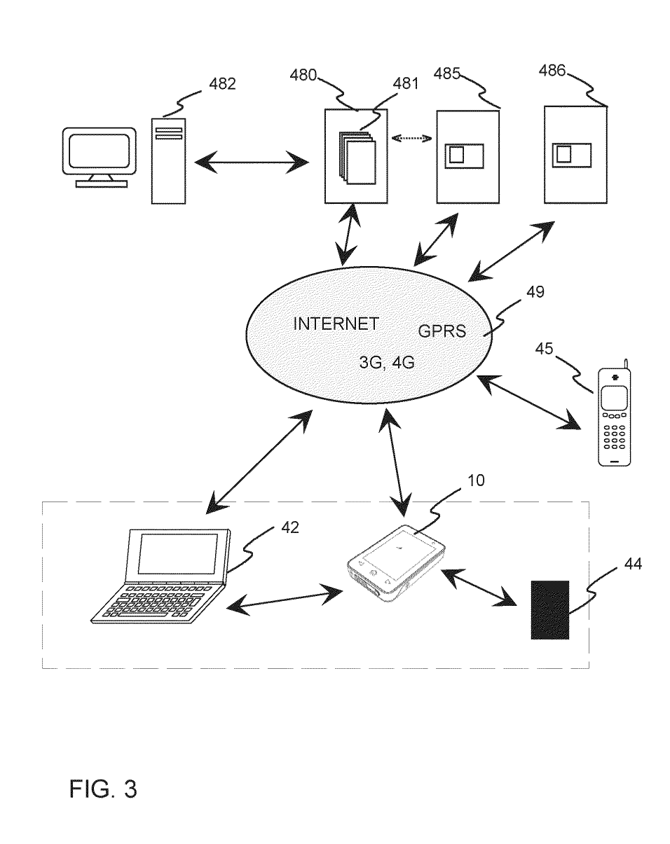

FIG. 2 illustrates a perspective view of an exemplary measurement device according to the invention. The measurement device has a large touch screen display 175, which displays the measurement result and possibly other reminders or activity instructions. It preferably shows the measurement result, reminders and/or other activity instructions as figures such as avatars. The device preferably also has audio means for producing sounds, voices and/or melodies. A sample input 24 and a USB connector 11 are located at the bottom side of the device. There is a sliding cover 21, which can be moved to cover either the sample input 24 or the electrical connection 11.

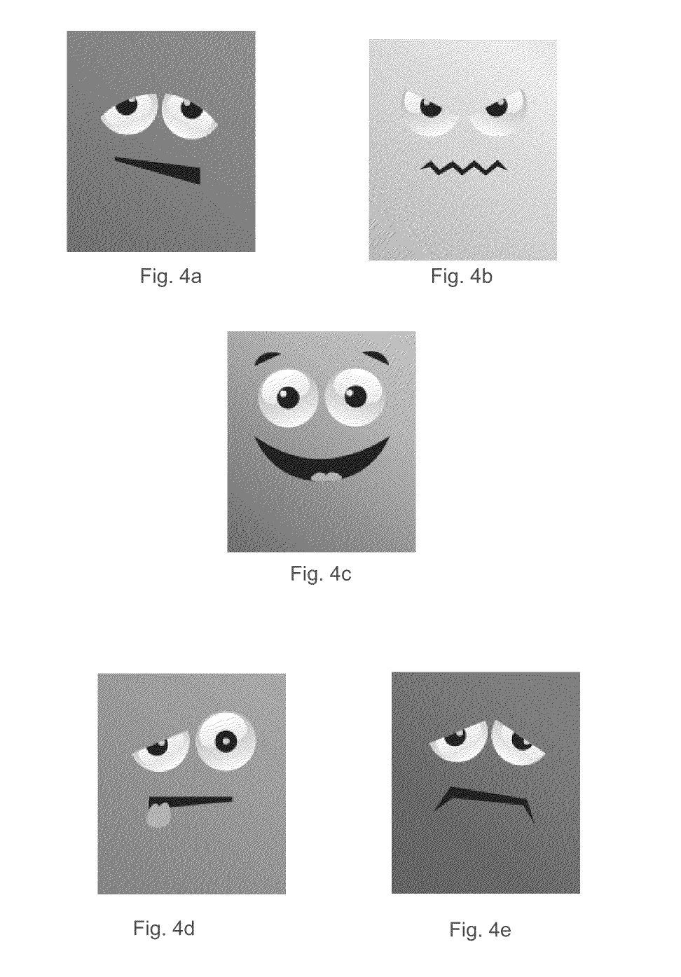

FIG. 3 illustrates a system where an exemplary measurement device 10 according to the invention is in connection with several types of equipment. The measurement device 10 can be connected to a user's laptop computer 42 with a wired, USB connection, in order to transfer measurement data and other user data, as well as update programs and parameters of the measurement device. The measurement device may also be in connection with other devices 44 of the user with a wired USB connection or wireless Bluetooth or NFC connection, for example. Such other devices may include other measurement devices, an insulin injector, etc. The measurement device preferably has a wireless connection with a cellular data transfer network in order to transfer data with mobile phones 45.

The measurement device may also be in connection with a public health care system. Data is transferred with a central unit 480 of a user information centre, using direct wireless communication through cellular data network, or using a computer 42 which is connected to the Internet. The above mentioned data may then be transferred between the measurement unit and the central unit. The central unit 480 comprises a database 481, into which personal information and information relating to the disease of the measurement device users is stored. The central unit can be e.g. a central computer of regional health care. The health care system includes terminal equipment 482 of the nursing staff, which terminal equipment is connected to the central computer 480. There may also be servers 485, 486 connected to the central computer, which servers may provide supplementary services for the users, such as games.

The central unit 480 can also be separate from other database of the health care, and which comprises only self-care information relating to a certain disease, such as information on blood glucose concentration measurement and care monitoring. Nursing staff, such as a nurse and a doctor, has access to the information of the central unit. The access may take place e.g. with terminal equipment 482 after logging in the system.

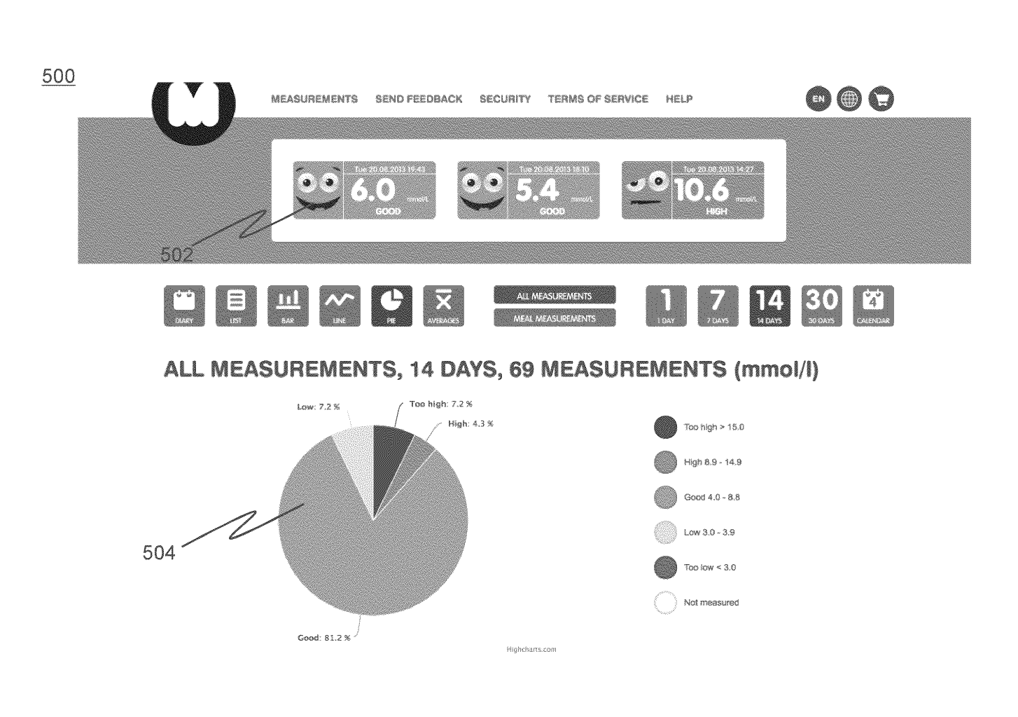

FIGS. 4a-4e illustrate exemplary character expressions coupled with a specific colors relating to different blood glucose concentration categories. Measured blood concentration is preferably classified into blood glucose concentration categories relating to how high or low the blood glucose concentration level is. In a preferable embodiment, there are five different blood glucose concentration categories in the device and the system of the present invention. When presenting the measurement result to the user on the display, the device utilizes means for coupling a numerical value with a character expression, a color and a sound relating to the specific blood glucose concentration category.

In FIG. 4a is illustrated an exemplary character expression coupled to an exemplary color presenting of too low blood glucose concentration level. Too low blood glucose concentration level, such as less than 3 mmol/l, can be presented with a very exhausted character expression and red color, for example. In addition, a sound that preferably represents a state, which requires an immediate action, coupled to the action of presenting a measurement result of too low blood glucose concentration category.

When presenting the measurement result to the user, the character expression in the color of too low blood glucose concentration category is presented on the display and, preferably, the related sound of that category is played at the same time. A numerical value of the measurement result is further presented on the display together with the other components, i.e. the character expression and the color, of too low blood glucose concentration category.

Respectively, FIGS. 4b represent an exemplary character expression and color of low blood glucose concentration level, such as values of 3 mmol/l or more, but less than 4 mmol/l. The appearance of the character expression can be selected to be not so exhausted than the appearance of the character expression of the too low blood glucose concentration category. Also the color can be selected to be more brightly, such as yellow, for indicating a better condition. An appropriate sound is also coupled to the other components of that blood glucose concentration category. Furthermore, FIG. 4c illustrates a character expression and a color of a blood glucose concentration category, which can be within 4.1-8.0 mmol/l. This is a desirable category and thus the character expression, the color and the sound of this category are preferably selected to indicate a good condition. The color representing this category can be e.g. green, which is normally used to relate to harmless situations.

The FIGS. 4d and 4e illustrate categories of high and too high blood glucose concentration category, respectively, which correspond values from 8,1 mmol/l to 15 mmol/l and more than 15 mmol/l. Because these two categories of high blood glucose concentration level are presenting harmful condition but in another way than low blood glucose concentration level, the selected colors for these categories should clearly differ from the categories of low blood glucose concentration level. An easy way to solve this problem is to allocate one part of the color map for low concentration categories, e.g. warm colors, and another for high concentration categories, e.g. cold colors.

As described above, an information or an suggestion for care action can be given on the display together with categories of 4a, 4b, 4d and 4e, such as suggesting food or insulin based on that particular blood glucose concentration category.

In an embodiment, the device and the system of the present invention comprise its own character expressions, but in another embodiment, different characters/expression categories are available to the user for selecting the characters/expressions he or she prefers, such as Angry Birds™ and Hello Kitty™. The person skilled in the art understands that there can be a vast number of character categories including games, cartoons, animated series and other toy series, for example.

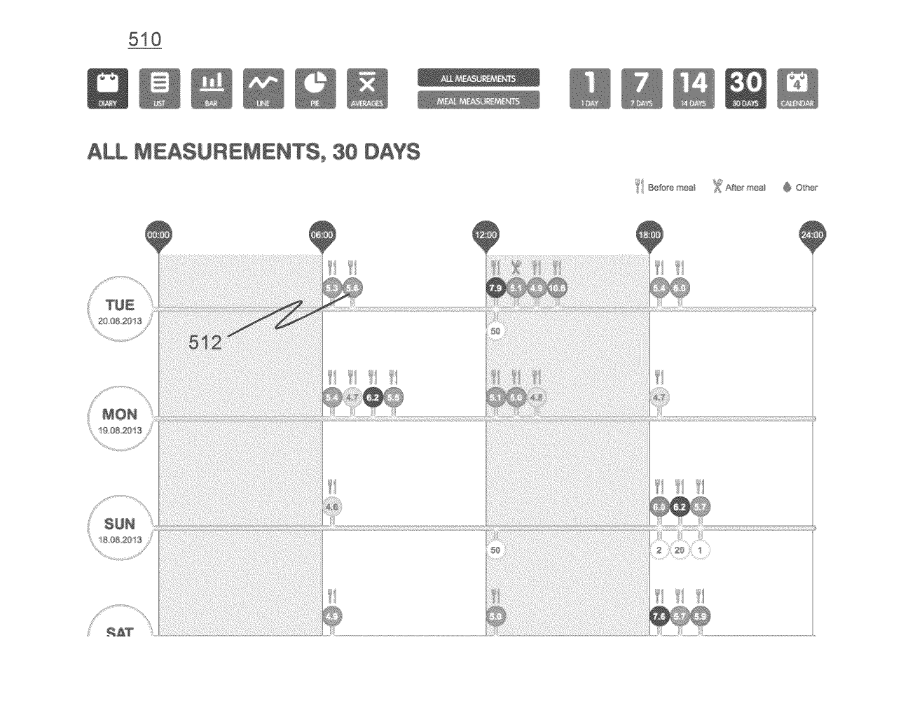

FIGS. 5a and 5b illustrate exemplary views of a user interface of the system according to the present invention. Measurement data in various time periods can be presented for the user. In a preferable embodiment, the measurement results have colors according to the category that they fall into. Also character expressions relating to the blood glucose concentration categories are presented together with using the color.

In FIG. 5a, the last three measurement results 502 with the related character expression and color are presented at the top of the view 500. The measurement data of selected time period can be present in several ways, such as one by one in diary, in list as bars, line or in a pie 504.

In FIG. 5b, the each measurement result 512 is presented in the diary view 510 preferably with category color and blood glucose concentration value. This view may provide a good tool to the user and other care taking personnel to monitor the results and to adjust the care.

The scope of the invention is determined by the attached claims together with the equivalents thereof. The skilled persons will again appreciate the fact that the explicitly disclosed embodiments were constructed for illustrative purposes only, and the scope will cover further embodiments, embodiment combinations and equivalents that better suit each particular use case of the invention.

1. A measurement and monitoring device for measuring and presenting blood glucose concentration comprising
measurement means for measuring blood glucose concentration, and
a user interface for presenting to a user information which is based on a measurement result, the user interface comprising at least one display and/or means for forming sound,
characterised in that the measurement and monitoring device comprises means for performing an action in order to present the measured blood glucose concentration result to the user, wherein the action comprises coupling of a character expression with a color, a sound and a numerical value.

2. The device according to claim 1, wherein blood glucose concentration results are classified into five different blood glucose concentration categories.

3. The device according to claim 2, wherein the presented character expression, color and sound are related to blood glucose concentration categories.

4. The device according to claim 1, wherein said character expression is selected from numerous character/expression categories.

5. The device according to claim 1 further comprising means for storing user specific data which relates to care of the user.

6. The device according to claim 1 further comprising means for providing the user with reminders and/or instructions.

7. The device according to claim 1 further comprising means for comparing a measurement result with said care data.

8. The device according to claim 1 further comprising means for recording user activities.
